(19) United States
(12) Patent Application Publication
(10) Pub. No.: US 2024/0149017 A1
(43) Pub. Date: May 9, 2024

(54) MULTI-WAY URETHRAL CATHETER

(71) Applicant: José António PACHECO DOS SANTOS DIAS, Lisboa (PT)

(72) Inventor: José António PACHECO DOS SANTOS DIAS, Lisboa (PT)

(21) Appl. No.: 18/531,796

(22) Filed: Dec. 7, 2023

Related U.S. Application Data
(63) Continuation-in-part of application No. 17/418,866, filed on Jun. 28, 2021, now abandoned.

(30) Foreign Application Priority Data
Dec. 27, 2018 (PT) 115232

(51) Int. Cl. A61M 25/00; A61M 25/10

(52) U.S. Cl. CPC A61M 25/0017; A61M 25/1018

(57) ABSTRACT

The present disclosure is enclosed in the area of multi-way bladder catheters, which are suitable for draining urine and/or blood and/or instillation liquid from the bladder of a patient. It refers to a bladder catheter (1) with a drainage outlet (5), a drainage channel (11) and drainage inlets (4), the drainage channel (11) connecting the drainage outlet (5) and the drainage inlets (4), and an instillation inlet (2), a branched channel (12) and more than one instillation outlets (3), the branched channel (12) having a portion with a single way connecting to the instillation inlet (2) and several portions each connecting to the instillation outlets (3), wherein the portion with a single way is furcated to each of the several portions connecting to an instillation outlet (3), such that instillation liquid injected in the instillation inlet (2) is furcated and ejected from each of the instillation outlets (3). It guarantees that no change of procedure is required for a practitioner, in a simplified manner.

### FIELD OF THE INVENTION

The present invention is enclosed in the area of multi-way or multi-channel bladder catheters, which are suitable for draining urine or blood from the bladder of a patient.

### PRIOR ART

The formation of clots within the bladder is a major problem in medicine. Clot formation occurs by the presence of blood within the bladder, either in catheterized or non-catheterized patients, after surgery (urological or otherwise) or in other contexts.

The causes of bleeding into the bladder are varied, from very frequent and serious tumours of the bladder, kidneys or ureters, to benign diseases, also very common, such as prostate diseases, lithiasis (urinary stones), infections, other diseases of the kidneys and ureters, systemic diseases like haematological disorders and secondary to several treatments and medicines, among others. This type of haemorrhage is almost universal in the postoperative period of multiple urological surgeries.

This problem occurs both in the hospital/inpatient setting and in the outpatient setting, outside the health facility setting (i.e., with outpatients), resulting in many patients having to be treated and catheterized as a result of bleeding.

Many are the cases in which patients are catheterized because they cannot urinate, but also as many are the other medical situations where such devices are required—for example, to measure the amount of urine produced. Presently, patients are being given medications to fight the formation of thrombi (which cause strokes), such as antiplatelet agents and anticoagulants. These drugs further contribute to urinary haemorrhage and an increased risk of clot formation and clogging of catheters.

An additional, unsolved, problem is the frequent and recurrent obstruction of the catheter (catheters for bladder drainage, to drain the urine inside this organ), specifically the clogging caused by clots in these catheters, which consist of a major cause of suffering to the catheterised patient, namely severe pain and discomfort due to bladder distension. This clogging significantly increases the risk of trauma to the urinary tract and infection, by the manipulation which the clearing of the clot in the catheter requires, namely by means of vesical washing, instillation of liquid into the bladder, replacements of catheter, etc.

This seemingly simple problem consumes countless resources, both from health professionals (doctors and nurses, operating assistants) and from equipment, devices and accessories. It consists, moreover, of a cause of pronounced suffering of the affected patients (by stopping the correct emptying of the bladder and sensation of repletion that is thereby caused), with intense pain.

This problem also increases the risk of hospital infection, the length of hospital stay, and of the number and severity of associated complications. By increasing the time patients are bedridden, the number of manoeuvres and manipulations related to the catheter (such as continuous and manual washes) triggers the risk of thromboembolic complications, infectious, traumatic complications for the urinary tract and consumption of medicaments, among others.

Catheters which provide active drainage of the bladder are known in the art, namely three-way catheters in which an instillation liquid is provided through the catheter and into the bladder, such liquid and the contents of the bladder then exiting the bladder through one or more drainage openings formed in the catheter tip and connected to another (drainage/outlet) way.

Thus, a first way of such catheters is used for introducing instillation liquid. The catheter thus has an inlet provided in one outer edge/proximal tip and connected to the instillation way, which then is connected to an outlet in the opposite edge—an inner edge/distal tip— of the catheter. The instillation way connects the instillation inlet to the instillation outlet. When the catheter is applied to a patient, the distal tip will be provided in the patient, will the proximal tip will be provided out, thereby being handled by a practitioner.

A second way of such catheters is used for draining urine from the bladder. The catheter thus has an inlet provided in the inner edge and connected to the drainage way, which then is connected to an outlet in the opposite edge—the outer edge—of the catheter. The drainage way connects the drainage inlet or inlets to the drainage outlet.

Additionally, they comprise a way for inflating a balloon, (which is also present in two-way catheters, which do not provide liquid instillation to the bladder, containing only the drainage way and the balloon way). An inlet is provided in the outer edge which is connected to the balloon inflation way, which then is connected to the balloon, which in turn is provided between the inner and outer edges of the catheter, closer to the inner edge. A fluid (e.g., sterile water) may be provided through the inflation inlet and the balloon is inflated, thus retaining the catheter inside the bladder until the balloon is deflated.

Typically, the catheter has an outer wall and the drainage way if provided centrally inside the wall, wherein it is surrounded by the wall. The wall has a certain thickness, and the instillation way is provided in the wall, inside such thickness. The section of the drainage way is typically larger than the section of any other way provided in the wall, such as the instillation way. The way connected to the balloon is, as the instillation way, provided in the wall of the balloon. In the inflation inlet, a valve is provided, to maintain the balloon inflated. The balloon way connects the inflation inlet to the balloon.

There also exist four-way balloons, which comprise duplicated ways. Thus, for instance, a second balloon may be provided, with the respective inflation inlet and valve, and a way connecting the second inflation inlet and the second balloon. In addition, a second drainage or instillation way may be provided. Thus, a second drainage/instillation outlet exists in the outer edge of the catheter, and a second drainage/instillation outlet in the inner edge of the catheter. This way connects the inner hole to the outside outlet/inlet hole.

The catheters known in the art thus allow to have a number of ways adapted to the desired application.

Still, the multi-way catheters having four or more ways have a particularly cumbersome usage, since they require a rather synchronized usage of the duplicated ways. For instance, a practitioner may have to actuate two instillation inlets simultaneously, such that two instillation outlets are simultaneously irrigating the bladder and the prostatic urethra. This activity requires either a great expertise of the practitioner or that several practitioners are performing the act. Otherwise, the effect of having two instillation ways is lost.

In an innovative manner, the present disclosure provides simplified solutions for such problems. It further provides a facilitated, continuous and abundant elimination of both liquid and any small clots that are present or formed in the bladder, whichever is the relative position of such elements, in particular clots, with regard to the catheter.

### SUMMARY OF THE INVENTION

The present disclosure refers to a urethral catheter which comprises:
a drainage outlet, a drainage channel and one or more drainage inlets, the drainage channel connecting the drainage outlet and the drainage inlet, and an instillation inlet, a branched channel and more than one instillation outlet, the branched channel having a portion with a single way connecting to the instillation inlet and several portions each connecting to an instillation outlet, providable inside a bladder, wherein the portion with a single way is furcated to each of the several portions connecting to an instillation outlet, such that instillation liquid injected in the instillation inlet is furcated and ejected from each of the instillation outlets.

The instillation inlet and the drainage outlet are provided in a first edge portion of the catheter and the instillation outlets and the (one or more) drainage inlet(s) are provided in a second edge portion of the catheter, opposite to the first edge portion, wherein the catheter may have an outer wall, the instillation outlets being provided along the edge portion and consisting of openings in the outer wall.

The instillation outlets and each corresponding portion of the branched channel connecting to an instillation outlet may be arranged such that instillation liquid is ejected perpendicularly to the outer wall.

An end of each portion of the branched channel connecting to an instillation outlet may be blocked, such portion of the branched channel thereby not extending beyond a corresponding instillation outlet, thereby distally to the respective instillation outlet.

The blockage of each of the several branched portions of the branched channel may be provided by means of beading, or by any other means.

Alternatively, the instillation outlets may be provided along the edge portion in a same position of the extension of the catheter, thereby circumferentially, such that the extensions of each of the several portions connecting to an instillation outlet are the same, thus instillation liquid being ejected at a same extension of the length of the catheter.

The drainage channel may be provided centrally and surrounded by an outer wall of the catheter having a thickness, the branched channel being provided inside the outer wall.

The several portions connecting to an instillation outlet may be evenly provided in the outer wall.

The instillation outlets may have a stadium, round or an oval shape.

The branched channel may be bifurcated, trifurcated or n-furcated, thereby correspondingly having two, three or n instillation outlets and correspondingly two, three or n portions connecting the single way portion to a corresponding instillation outlet, wherein n is an integer higher then 3.

The instillation inlets may be provided at the tip of the second edge portion, such that they are provided perpendicularly to the outer wall. The catheter may comprise a plurality of instillation inlets of different shapes connected to the drainage way.

The catheter comprises an inflation inlet, an inflation channel and a balloon, the inflation channel connecting the inflation inlet to the balloon, such that a fluid provided through the inflation inlet inflates the balloon, the catheter further comprising a valve provided between the inflation inlet and the inflation channel, the valve having a minimum pressure threshold which prevents the balloon from deflating.

The balloon may be provided between the first edge portion and the second edge portion, closer to the second edge portion.

The outer wall may be made of latex, silicone or a silicone polymer, optionally an inert silicone polymer.

### DESCRIPTION OF FIGURES

FIG. 1—a side view of a catheter (1) according to the present disclosure.

FIG. 2—a cross-section view of a catheter (1) according to the present disclosure, in the part of the catheter in which the furcated portions are provided.

FIG. 3—a view from perspective of a catheter (1) according to the present disclosure.

### DETAILED DESCRIPTION

The present disclosure refers to a bladder catheter.

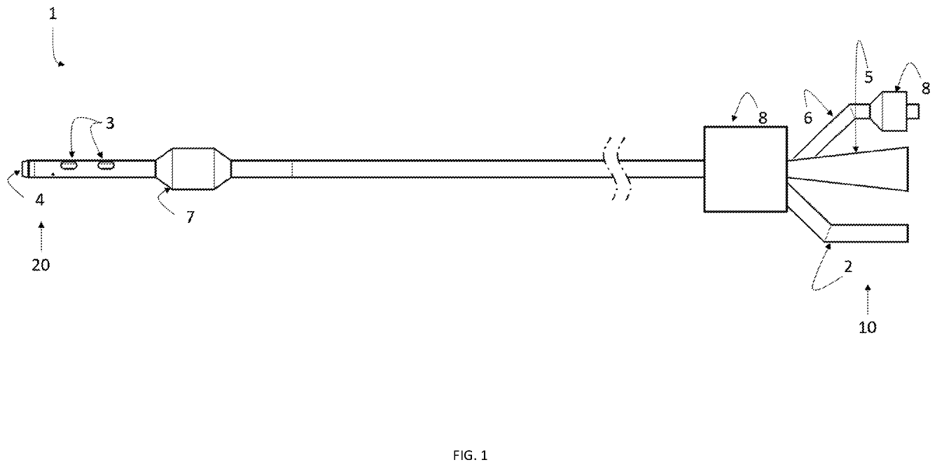

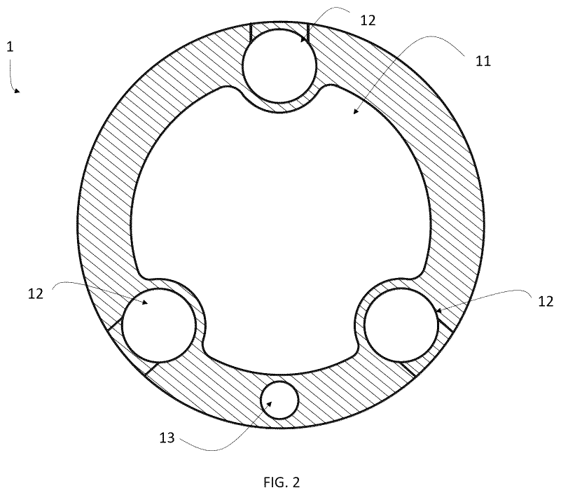

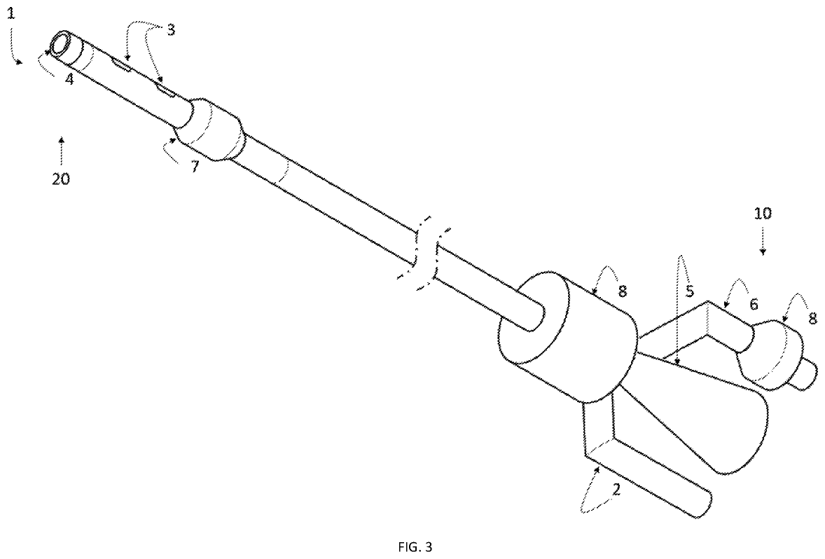

FIGS. 1-3 are used to refer to the several elements of the bladder catheter.

It comprises a drainage outlet (5), a drainage channel (11) and a drainage inlet (4) (or more), the drainage channel (11) connecting the drainage outlet (5) and the drainage inlet (4).

These three elements, which are directly connected to each other, provide the part of a circuit formed by the catheter (1) which removes urine and/or blood from the bladder. Upon instillation or irrigation of the bladder, the instillation liquid runs across the inside of the bladder and mixes with its content. The mixed liquid will then enter the drainage inlet (4), cross the drainage channel (11) and exit the catheter (1) via the drainage outlet (5), provided at the end of the catheter, in an outer edge. The drainage inlet (4) is provided at an inner edge/distal tip or close to an inner edge, which comprises the so called insertion tip of the catheter.

The catheter (1) further comprises an instillation inlet (2), a branched channel (12) and more than one instillation outlet (3), providable inside the bladder of a patient.

Instillation liquid injected in the instillation inlet (2) crosses the branched channel (12) and is ejected from each of the instillation outlets (3).

By comprising more than one instillation outlet (3), the catheter (1) provides that instillation liquid better spreads through the bladder.

The branched channel (12) has a portion with a single way connecting to the instillation inlet (2). In such portion of the branched channel (12), a cross-section of the catheter (1) will show a way corresponding to the drainage channel (11) (preferably provided centrally and surrounded by an outer wall) and the single way of the branched channel (12) [preferably in the outer wall, thus peripherally as regards the drainage channel (11)].

The branched channel (12) further has several portions, each portion connecting to an instillation outlet (3), wherein the branched channel (12) is such that the portion with a single way is furcated to each of the several portions connecting to an instillation outlet (3). Thus, a cross-section of the catheter (1) after the furcation will show a way corresponding to the drainage channel (11) (preferably provided centrally and surrounded by an outer wall) and the more than one way of the branched channel (12) [preferably in the outer wall, thus peripherally as regards the drainage channel (11)]. A furcation may be provided in a furcation element (8), an element of the catheter (1) which furcates the single way of the branched channel (12) into two or more ways.

A cross-section of the catheter (1) at the furcation will again show a way corresponding to the drainage channel (11) (preferably provided centrally and surrounded by an outer wall) and the furcation from the single way to the more than one way of the branched channel (12) [preferably in the outer wall, thus peripherally as regards the drainage channel (11)].

The branched channel (12) and the respective furcation are so configured that instillation liquid injected in the instillation inlet (2) is furcated and ejected from each of the instillation outlets (3). The route that the instillation liquid follows is thus the following: being injected in a single instillation inlet (2), crossing the single way of the branched channel (12), being furcated inside the branched channel (12), crossing each furcated portion of the branched channel (12) and exiting the catheter (1) via each of the instillation outlets (3), into the bladder and mixing with its content. The drainage process previously described then follows.

As previously referred, prior art multi-way catheters have a particularly cumbersome usage, since by having more than one instillation opening, they require the simultaneous actuation of the several openings. The catheter (1) of the present disclosure is a multi-way catheter, having several ways provided in it due to the furcation of the branched channel (12) and thereby providing the injection of instillation liquid by means of the several instillation outlets (3), but at the same way also behaving as a single way for instillation, from the point of view of its operation by a practitioner. This means that it guarantees that no change of procedure is required, since the drainage outlet (5) and the single instillation inlet (2) are exactly the same for a regular prior art catheter, as those previously described.

Thus, a practitioner using the catheter (1) of the present disclosure can irrigate the bladder by means of several instillation openings operating a single instillation inlet (2), thereby being of a much easier operation when compared with prior art multi-way catheters, while keeping a simplified construction.

The instillation inlet (2) and the drainage outlet (5) are provided in a first edge portion (10) of the catheter (1) and the instillation outlets (3) and the drainage inlet (4) are provided in a second edge portion (20) of the catheter, opposite to the first edge portion (10). The first edge portion (10) corresponds to a so-called outer edge portion or proximal tip, the portion of the catheter (1) which the practitioner handles (outside the patient) when the catheter (1) is provided inside a patient. The second edge portion (20) corresponds to a so-called inner edge portion or distal tip, the portion of the catheter (1) which comprises the insertion tip and which is provided inside a patient when in use.

The catheter (1) may have an outer wall, wherein the instillation outlets (3) are provided along the edge portion and consist of openings in the outer wall. The instillation outlets (3) and each corresponding portion of the branched channel (12) connecting to an instillation outlet (3) may be arranged such that instillation liquid is ejected perpendicularly to the outer wall.

Such arrangement provides a facilitated, continuous and abundant movement of the content inside the bladder (urine, blood and instillation liquid) and eases and facilitates the subsequent elimination of both liquid and any small clots that are present or formed in the bladder, whichever is the relative position of such elements, in particular clots, with regard to the catheter. As previously referred, the catheter (1) allows for a practitioner to actuate a single instillation inlet (2). In addition, the several distributed instillation outlets (3), which eject instillation liquid perpendicularly to the outer wall, allow for the liquid to better distribute and run across the bladder, thereby better mixing with its content, and allowing a better drainage.

An end of each portion of the branched channel (12) connecting to an instillation outlet (3) may be blocked, in a way that the portion of the branched channel (12) does not extend beyond a corresponding instillation outlet (3). Such blockage enables that the pressure with which the instillation liquid runs inside each furcated or branched portion is fully and directly transferred to the ejection, by means of the blockage, which does not extend beyond the respective outlet.

The blockage of each of the several branched portions of the branched channel (12) may be provided by means of beading, or by any other means, of the branched channel (12) beyond the instillation outlet (3). The branched channel (12) may thus be formed along the extension of the catheter (1) between the furcation and the insertion tip, wherein after the instillation outlet (3) it is blocked by beading, or by any other means.

The instillation outlets (3) may be provided along the edge portion in different positions of the extension of the catheter. This allows for a better distribution of instillation liquid throughout the bladder. FIG. 1 shows these different positionings. The furcated portions of the branched channel (12) extend until each of the instillation outlets (3).

The instillation outlets (3) may be provided along the edge portion in a same position of the extension of the catheter, such that the extensions of each of the several portions connecting to an instillation outlet (3) are the same, thus instillation liquid being ejected at a same extension of the length of the catheter.

The drainage channel (11) may be provided centrally and surrounded by an outer wall of the catheter (1) having a thickness, the branched channel (12) being provided inside the outer wall, wherein this arrangement provides a higher simplicity of construction (and an adequate drainage capability of the bladder content), which is particularly suitable for the furcation of the branched channel (12). The single way portion, the furcation and the several furcated portions are provided in the outer wall, within its thickness.

The several portions connecting to an instillation outlet (3) may be evenly provided in the outer wall. Thus, for instance, if two instillation outlets (3) and two corresponding furcated portions of the branched channel (12) are provided, a cross-section of the catheter (1) in the furcated portions part will show the drainage channel (11) provided centrally and two channels provided inside the outer wall, the two channels corresponding to the furcated portions of the branched channel (12), and such channels being equidistantly provided. If three instillation outlets (3) and three corresponding furcated portions of the branched channel (12) are provided, a cross-section of the catheter (1) in the furcated portions part will show the drainage channel (11) provided centrally and three channels provided inside the outer wall, the three channels being equidistantly provided in the wall, the distance (and angle, 120′) between each two being the same. This arrangement enhances the distribution of instillation liquid inside the bladder, making the effectiveness of the catheter (1) independent of its positioning in the patient. For instance, it could happen that a certain placement would result in an area not being irrigated, as to such area would not correspond an instillation outlet (3). This typically occurs when the patient is laying down or seated. With such equidistant arrangement, all sections/parts of the bladder will better be run across with instillation liquid, assuring the most efficacious induction of movement inside the bladder, avoiding clot formation and its serious consequences and complications.

The instillation outlets (3) may have a stadium, a round, an oval, or other shape.

The branched channel (12) may be trifurcated, thereby having three instillation outlets (3) and three portions connecting the single way portion to a corresponding instillation outlet (3).

Each instillation inlet (2) may be provided at the tip of the second edge portion (20)— the edge of the insertion tip such that it is provided perpendicularly to the outer wall.

The catheter (1) comprises an inflation inlet (6), an inflation channel (13) and a balloon (7), the inflation channel (13) connecting the inflation inlet (6) to the balloon (7), such that a fluid provided through the inflation inlet (6) inflates the balloon (7), the catheter (1) further comprising a valve (14) provided between the inflation inlet (6) and the inflation channel (13), the valve (14) having a minimum pressure threshold which prevents the balloon (7) from deflating.

Thus, to the inflation channel (13), a further way will be provided. This means that, when a cross-section of the catheter (1) in the part comprising the single way portion of the branched channel (12) will show a way corresponding to the drainage channel (11) (preferably provided centrally and surrounded by an outer wall), the single way of the branched channel (12) [preferably in the outer wall, thus peripherally as regards the drainage channel (11)], and the single way of the inflation channel (13) [preferably in the outer wall, thus also peripherally as regards the drainage channel (11)].

The balloon (7) may be provided between the first edge portion (10) and the second edge portion (20), closer to the second edge portion (20). In particular, the balloon (7) may be provided in a part of the catheter (1) in which the branched channel (12) has several furcated portions. Thus, the furcation of the branched channel (12) is closer to the outer edge—being closer to the outer end—than the balloon (7). The balloon (7) may also be provided between the first edge portion (10) and the second edge portion (20), closer to the first edge portion (10) and proximally [closer to the first edge portion (10)] to/than the furcation.

The outer wall may be made of latex, silicone or a silicone polymer, optionally an inert silicone polymer.

Such constructional elements allow, further to the described advantages, to provide a catheter (1) with the same external gauge as other multi-way catheters known in the art. For instance, a catheter (1) according to the present disclosure with three furcated portions of the branched channel (12) has a same external gauge as a three-instillation channel catheter (1) known in the art. This is also provided while the drainage channel (11) is particularly wide.

As will be clear to one skilled in the art, the present invention should not be limited to the embodiments described herein, and a number of changes are possible which remain within the scope of the present invention.

Of course, the preferred embodiments shown above are combinable, in the different possible forms, being herein avoided the repetition all such combinations.

1. A bladder catheter (1) characterised in that it comprises:
a drainage outlet (5), a drainage channel (11) and one or more drainage inlets (4), the drainage channel (11) connecting the drainage outlet (5) and the drainage inlet,
the catheter (1) further comprising:
an instillation inlet (2), a branched channel (12) and more than one instillation outlets (3), the branched channel (12) having a portion with a single way connecting to the instillation inlet (2) and several portions each connecting to an instillation outlet (3), providable inside a bladder wherein the portion with a single way is furcated to each of the several portions connecting to an instillation outlet (3), such that instillation liquid injected in the instillation inlet (2) is furcated and ejected from each of the instillation outlets (3).

2. The catheter (1) according to claim 1 wherein the instillation inlet (2) and the drainage outlet (5) are provided in a first edge portion (10) of the catheter (1) and the instillation outlets (3) and the drainage inlet are provided in a second edge portion (20) of the catheter, opposite to the first edge portion (10),
wherein the catheter (1) has an outer wall, the instillation outlets (3) being provided along the edge portion and consisting of openings in the outer wall.

3. The catheter (1) according to claim 2 wherein the instillation outlets (3) and each corresponding portion of the branched channel (12) connecting to an instillation outlet (3) are arranged such that instillation liquid is ejected perpendicularly to the outer wall.

4. The catheter (1) according to claim 3 wherein an end of each portion of the branched channel (12) connecting to an instillation outlet (3) is blocked, such portion of the branched channel (12) thereby not extending beyond a corresponding instillation outlet (3).

5. The catheter (1) according to claim 4 wherein the blockage of each of the several branched portions of the branched channel (12) distally to the instillation outlets (3) is provided by means of beading, or by any other means.

6. The catheter (1) according to claim 2, wherein the instillation outlets (3) are provided along the edge portion in a same position of the extension of the catheter, such that the extensions of each of the several portions connecting to an instillation outlet (3) are the same, thus instillation liquid being ejected at a same extension of the length of the catheter.

7. The catheter (1) according to claim 1 wherein the drainage channel (11) is provided centrally and surrounded by an outer wall of the catheter (1) having a thickness, the branched channel (12) being provided inside the outer wall.

8. The catheter (1) according to claim 7 wherein the several portions connecting to an instillation outlet (3) are evenly provided in the outer wall.

9. The catheter (1) according to claim 1 wherein the instillation outlets (3) have a stadium, a round, or an oval shape.

10. The catheter (1) according to claim 1 wherein the branched channel (12) is bifurcated, trifurcated or n-furcated, thereby having two, three or n instillation outlets (3) and two, three or n portions connecting the single way portion to a corresponding instillation outlet (3), n consisting of an integer higher than 3.

11. The catheter (1) according to claim 2 wherein each instillation inlet (2) is provided at the tip of the second edge portion (20), such that it is provided perpendicularly to the outer wall.

12. The catheter (1) according to claim 1 wherein it further comprises an inflation inlet (6), an inflation channel (13) and a balloon (7), the inflation channel (13) connecting the inflation inlet (6) to the balloon (7), such that a fluid provided through the inflation inlet (6) inflates the balloon (7), the catheter (1) further comprising a valve (14) provided between the inflation inlet (6) and the inflation channel (13), the valve (14) having a minimum pressure threshold which prevents the balloon (7) from deflating.

13. The catheter (1) according to claim 12 wherein the balloon (7) is provided between the first edge portion (10) and the second edge portion (20), closer to the second edge portion (20).

14. The catheter (1) according to claim 1 wherein the outer wall is made of latex, silicone or a silicone polymer, optionally an inert silicone polymer.
